(19) United States
(12) Patent Application Publication
(10) Pub. No.: US 2006/0047335 A1
(43) Pub. Date: Mar. 2, 2006

(54) Catheter with deflector

(76) Inventor: Henry M. Israel, Bnei Brak (IL)

Correspondence Address: Dekel Patent Ltd.;Beit HaRofim, Room 27, 18 Menuha VeNahala Street, Rehovot, IL

(21) Appl. No.: 10/925,979

(22) Filed: Aug. 26, 2004

(51) Int. Cl. A61F 2/06

(52) U.S. Cl. 623/1.11

(57) ABSTRACT

A stent assembly including a catheter formed with a side aperture, and a deflector positioned near the side aperture adapted to deflect a guidewire pushed thereagainst to pass through the side aperture.

### FIELD OF THE INVENTION

The present invention relates generally to stents, and particularly to a catheter with structure to redirect a guidewire that facilitates implanting bifurcated stents in a body.

### BACKGROUND OF THE INVENTION

A stent is a well known device used to support an intraluminal wall, used in procedures, such as but not limited to, percutaneous transluminal coronary angioplasty (PTCA). Various types of stent architectures are known in the art, including braided stents (filaments or wires, wound or braided into a particular configuration), or mesh stents (metal mesh bent or formed into a particular shape), among others.

Typically, a stent may be restrained in a radially compressed configuration by a sheath or catheter, and delivered by an introducer to the site where it is required. The introducer may pass over a guidewire (like a monorail) that has been entered through the patient's skin, or through a blood vessel exposed by minor surgical means. When the introducer has been threaded into the body lumen to the stent deployment location, the introducer is manipulated to cause the stent to be released. The stent expands to a predetermined diameter at the deployment location, and the introducer is withdrawn. Stent expansion may be effected by spring elasticity, balloon expansion, or by the self-expansion of a thermally or stress-induced return of a shape memory alloy (such as a nickel-titanium alloy, e.g., NITINOL) to a pre-conditioned expanded configuration.

There are bifurcated lumens, such as but not limited to, the carotid artery, which may need support with a bifurcated stent. A bifurcated lumen (also called bifurcation) is an area of the vasculature where a first vessel is bifurcated into two or more branch vessels. Stenotic lesions may form in or around such bifurcations, that is, in or around one or more of the vessels.

However, delivering and deploying a stent to support a bifurcated lumen is a difficult challenge. Some of the problems include the difficulty of properly orienting the stent with respect to the bifurcation and the difficulty of providing a stent that supports the main trunk and branches of the bifurcation without blocking the passageways or causing turbulence or other flow disruptions.

PCT patent application PCT/IL03/00814 to Henry Israel, describes a bifurcated stent assembly with a stent sheath that includes two individually removable portions, one removable in a distal direction and the other in a proximal direction.

U.S. Pat. No. 6,494,905 to Zedler et al. describes a balloon catheter for use in the region of a vessel branching and, in particular, in coronary vessels. The catheter comprising a catheter stem disposed at the distal end of which are provided at least one balloon and at least one first guide means arranged in the region thereof for positioning the balloon in the region of a vessel branching. The first guide means is adapted to be introduced into the lateral branch of the vessel transversely with respect to the longitudinal direction of the balloon catheter. The balloon comprises at least two chambers which are spaced from each other in the longitudinal direction of the balloon catheter and between which the first guide means is arranged

U.S. Pat. No. 6,761,734 to Suhr describes a segmented balloon catheter for use in treating a condition of a blood vessel occurring near a bifurcation. The catheter comprises a shaft which includes a proximal end, a distal end and a longitudinal passageway that extends therethrough from the proximal end to the distal end. A first balloon portion is mounted on the shaft adjacent the distal end, and a second balloon portion is mounted on the shaft adjacent the first balloon portion. The shaft also comprises a transverse port which extends between the longitudinal passageway and the exterior of the segmented balloon catheter from between the first and second balloon portions. In this manner, a proximal end of a first guide wire which is pre-positioned in the main vessel may be inserted into the distal end of the shaft and threaded through the longitudinal passageway and out the proximal end of the shaft, and a proximal end of a second guide wire which is pre-positioned in the side branch vessel may be inserted into the transverse port and threaded through the longitudinal passageway and out the proximal end of the shaft.

A problem of the prior art stent assemblies is that the stent/catheter must be slid over a double guidewire (i.e., a pair of guidewires). At some point along the travel over the guidewires, the stent/catheter tends to twist and bind or otherwise get snagged, hindering the travel of the stent to the desired site.

### SUMMARY OF THE INVENTION

The present invention seeks to provide a catheter configuration that facilitates implanting bifurcated stents in a body, and which may be useful in deploying a guidewire through a side aperture of a main stent into a bifurcation of a blood vessel, for example. In one embodiment, a main stent may be deployed over the guidewire, and then the same guidewire may be redirected through a side aperture of the catheter to enable deploying a branch stent through that aperture. This obviates the need for working with two guide wires or trying to push a second guidewire through the side aperture.

There is provided in accordance with an embodiment of the present invention a stent assembly including a catheter formed with a side aperture, and a deflector positioned near the side aperture adapted to deflect a guidewire pushed thereagainst to pass through the side aperture.

In accordance with an embodiment of the present invention, the deflector may include a flap formed in a side wall of the catheter. The deflector may be spaced from an inner side wall of the catheter by a gap. A guidewire may be disposed in the catheter, wherein pushing the guidewire against the deflector deflects the guidewire through the side aperture.

In accordance with another embodiment of the present invention, the deflector may be directionally resilient to permit a guidewire to pass therethrough in one direction only. In such an embodiment, the deflector acts like a one-way valve. Main and branch stents may be assembled with the catheter and introduced over the same guidewire.

### BRIEF DESCRIPTION OF DRAWINGS

The present invention will be further understood and appreciated from the following detailed description taken in conjunction with the drawing in which:

FIG. 1 is a simplified pictorial illustration of a stent assembly, constructed and operative in accordance with an embodiment of the invention, including a catheter introduced into a body lumen over a guidewire, and including a deflector near a side aperture of the catheter;

FIG. 2 is an enlarged illustration of the deflector of the catheter of FIG. 1;

FIG. 3 is a simplified pictorial illustration of the guidewire being pulled to the proximal side of the deflector;

FIG. 4 is a simplified pictorial illustration of the guidewire being pushed against the deflector and being deflected into a branch lumen of the bifurcation; and

FIG. 5 is a simplified illustration of introducing a branch stent over the branch guidewire and through the side aperture, in accordance with an embodiment of the invention.

### DETAILED DESCRIPTION OF EMBODIMENTS

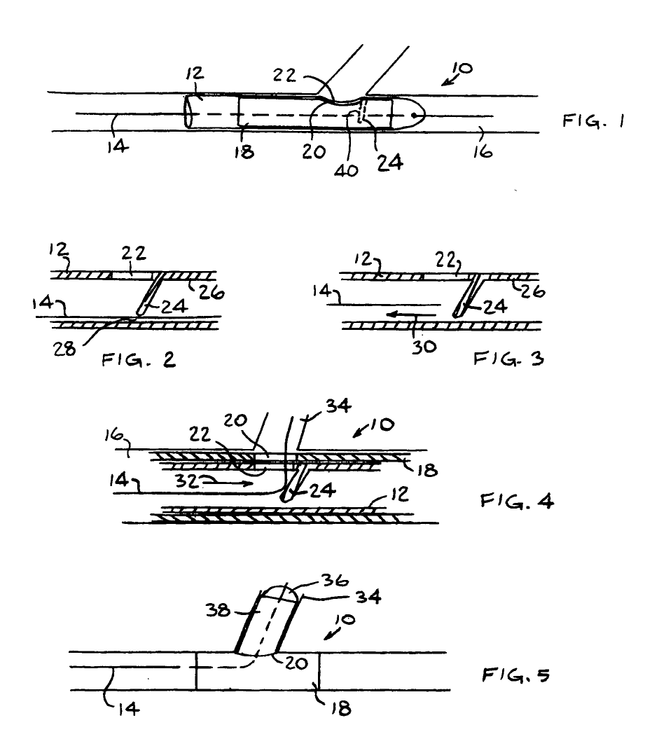

Reference is now made to FIG. 1, which illustrates a stent assembly 10, constructed and operative in accordance with an embodiment of the invention. Stent assembly 10 may include a catheter 12, which may be passed over a guidewire 14 to enter a body lumen 16, such as but not limited to a blood vessel. As is well known in angioplasty, guidewire 14 may be introduced by means of an introducer sheath (not shown) inserted through some lumen (e.g., femoral artery), and guidewire 14 is manipulated through vasculature to the site of implanting the stent. Guidewire 14 may be grasped and manipulated (e.g., pushed, pulled, twirled and twisted) at its proximal end, and may be constructed of any suitable material for guidewires, as is well known in the art.

A main stent 18 may be initially disposed in catheter 12 in a contracted orientation prior to deployment (this option shown in FIG. 1), and slides over guidewire 14 together with catheter 12. In such an embodiment, the main stent 18 is a self-expanding stent, which may be constructed from a suitable material, such as but not limited to, a shape memory alloy (such as a nickel-titanium alloy, e.g., NITINOL). Main stent 18 may be a wire mesh or braided stent, or any other kind of stent, but the invention is not limited to this construction or to self-expanding stents. For example, without limitation, stent 18 may be balloon-expandable, constructed from a suitable material, such as but not limited to, stainless steel 316L. In such an embodiment, the stent 18 is disposed over the catheter 12 (this option shown in FIG. 4). In general, stent 18 is “assembled with” the catheter 12, which encompasses self-expanding, balloon-expandable and any other kinds of stents. Stent 18 may be coated, such as a drug-eluting stent that has a polymer coating that emits an anti-restenosis drug. Stent 18 may be formed with a side opening 20 and catheter 12 may be formed with a side aperture 22 for placing therethrough a branch stent, as is described hereinbelow.

Reference is now made additionally to FIG. 2. In accordance with an embodiment of the invention, catheter 12 may include a deflector 24 that enables deflecting guidewire 14 through side aperture 22, as is explained further below. In one non-limiting embodiment, deflector 24 may be constructed by laser cutting or otherwise forming a flap in a side wall 26 of catheter 12. The flap may be bent into the catheter and becomes deflector 24. The opening left in the side wall 26 becomes side aperture 22. The area may be heat treated as necessary. Of course, the invention is not limited to this construction and the deflector 24 may be formed in other ways from other materials. In general, deflector 24 may be constructed of any metal, plastic or other materials that are medically safe.

Deflector 24 may be sized and shaped so that deflector 24 is spaced from the inner side wall 26 by a gap 28. Guidewire 14 may initially pass through gap 28, as seen in FIG. 2, wherein catheter 12 slides over guidewire 14 to the site of a bifurcation, and the main stent 18 is positioned at the bifurcation.

Referring to FIG. 3, guidewire 14 may be pulled proximally in the direction of an arrow 30 to be on the proximal side of deflector 24. As seen in FIG. 4, guidewire 14 may then be pushed distally in the direction of an arrow 32 against deflector 24. Deflector 24 deflects the guidewire 14 so that it passes through side opening 20 of main stent 18 and through side aperture 22 of catheter 12 into a branch lumen 34 of the bifurcation.

Reference is now made to FIG. 5. Another catheter 36 may be slid over guidewire 14. A branch stent 38 may be initially disposed in catheter 36 in a contracted orientation prior to deployment, and slides over guidewire 14 together with catheter 36. As described hereinabove for main stent 18, branch stent 38 may be a wire mesh or braided stent, or any other kind of stent, e.g., balloon-expandable or self-expanding, and may be coated, such as a drug-eluting stent. The branch stent 38 may be introduced through the side opening 20 of main stent 18 into the branch lumen 34. Upon withdrawal of catheter 36, branch stent 38 may expand or be otherwise deployed in branch lumen 34.

It is noted that the terms “push” and “pull” are relative terms and encompass any suitable motion and are not limited to the strict sense of push or pull. Also, the terminology of “sliding” a catheter over a guidewire is meant to encompass any kind of motion of the catheter with respect to the guidewire, such as but not limited to, monorail fashion.

Additionally or alternatively, deflector 24 may be directionally resilient or flexible (the terms being used interchangeably) to permit guidewire 14 to pass therethrough in one direction only, e.g., only in the proximal direction (arrow 30) not in the distal direction (arrow 32). Deflector 24 thus acts like a one-way valve (e.g., deflector 24 may be constructed as a spring-loaded flap). In such an embodiment there is no need for gap 28, rather the guidewire 14 can move proximally through the deflector 24, but when pushed distally against the deflector 24 gets deflected through the side aperture 22. The directional resilience of deflector 24 may be controlled through choice of materials, heat treatment and geometry, for example. Of course, it is appreciated that the invention may alternatively be carried out wherein the deflector 24 permits guidewire 14 to pass through distally and not proximally.

As another alternative, shown in FIG. 1, deflector 24 may be formed with a hole 40 through which guidewire 14 may originally pass. As described above, guidewire 14 may be pulled proximally to the proximal side of deflector 24 and then pushed distally against deflector 24.

The invention may be carried out with any kind of stent, such as but not limited to, the balloon catheters shown in U.S. Pat. No. 6,494,905 to Zedler et al. or U.S. Pat. No. 6,761,734 to Suhr.

It is appreciated that various features of the invention which are, for clarity, described in the contexts of separate embodiments, may also be provided in combination in a single embodiment. Conversely, various features of the invention which are, for brevity, described in the context of a single embodiment, may also be provided separately or in any suitable subcombination.

What is claimed is:

1. A stent assembly comprising:
a catheter formed with a side aperture; and
a deflector positioned near the side aperture adapted to deflect a guidewire pushed thereagainst to pass through said side aperture.

2. The stent assembly according to claim 1, wherein said deflector comprises a flap formed in a side wall of said catheter.

3. The stent assembly according to claim 1, wherein said deflector is spaced from an inner side wall of said catheter by a gap.

4. The stent assembly according to claim 1, further comprising a guidewire disposed in said catheter, wherein pushing said guidewire against said deflector deflects said guidewire through said side aperture.

5. The stent assembly according to claim 1, wherein said deflector is directionally resilient to permit a guidewire to pass therethrough in one direction only.

6. The stent assembly according to claim 1, wherein said deflector is formed with a hole adapted for a guidewire to pass through.

7. The stent assembly according to claim 1, further comprising a main stent assembled with said catheter.

8. The stent assembly according to claim 1, further comprising a branch stent assembled with said guidewire.
